(19) United States
(12) Patent Application Publication
(10) Pub. No.: US 2016/0170445 A1
(43) Pub. Date: Jun. 16, 2016

(54) MEASURING DEVICE CONFIGURED TO BE WORN ON THE WRIST OF A USER

(71) Applicant: National Electronics & Watch Co., LTD., Aberdeen (HK)

(72) Inventor: Kwong Yuen WAI, Aberdeen (HK)

(21) Appl. No.: 14/149,801

(22) Filed: Jan. 7, 2014

Related U.S. Application Data
(60) Provisional application No. 61/749,910, filed on Jan. 7, 2013.

(51) Int. Cl. G06F 1/16; G06F 1/18; A61B 5/00; H01R 24/28

(52) U.S. Cl. CPC G06F 1/163; H01R 24/28; G06F 1/1679; G06F 1/182; G06F 1/1637; A61B 5/6824

(57) ABSTRACT

Measuring devices can be configured to be worn on a wrist of a user for sensing data from the user. A measuring device can include a processor, a memory, and a case including a display and the processor. The measuring device can also include aUSB plug electrically and mechanically coupled with and extending from the case, the USB plug structured and arranged to be insertable directly into a USB port of a device, the USB plug including a first coupling member. The measuring device can include first and second straps configured to secure the measuring device to the wrist of the user, the first strap including a second coupling member configured to be coupled with the first coupling member. Moreover, the measuring device can be configured to measure a physical parameter of the user via a sensor on the measuring device.

### CROSS-REFERENCE TO RELATED APPLICATION

This application claims the benefit of priority of U.S. provisional patent application Ser. No. 61/749,910, filed on Jan. 7, 2013, the contents of which are incorporated herein by reference in their entirety.

### TECHNICAL FIELD

The present disclosure relates to a measuring device carried by a user during exercise for measuring non-invasively at least one signal from the body. More particularly, the disclosure is directed to a device configured to be worn on a user's wrist.

### BACKGROUND

Various portable personal measuring devices for measuring a signal of the user's choice from the body have been designed during the last few years. Devices have been designed for different end users: persons concerned with their health, fitness enthusiasts, goal-oriented athletes, and sports champions.

Signals to be measured include, for example, heart rate and arterial blood pressure. These measurements can be carried out non-invasively, i.e. the measuring sensors are disposed on a person's skin. Hence the use of such measuring sensors is safe and suitable for everyone.

A measuring device designed for measuring heart rate, i.e. a heart rate monitor, for example, is employed to improve physical and mental condition efficiently and safely. The user can employ a heart rate monitor to monitor his heart rate level during exercise, for example, and avoid excessive stress. A heart rate monitor can also be utilized in slimming since it has been scientifically shown that the most efficient way to burn fat stored in the body is to exercise at a given heart rate (about 55 to 65%) of a person's maximum heart rate. The maximum heart rate is calculated e.g. by subtracting the person's age from 220, or the maximum heart rate can also be measured.

U.S. Pat. No. 4,625,733 to Saynajakangas teaches a wireless and continuous heart rate measuring concept employing a transmitter attached to a user's chest for ECG accurate measuring of the user's heart rate and for telemetric transfer of heart rate data to a heart rate receiver attached to the user's wrist by employing magnetic coils in the transfer.

In addition to a receiver, the unit attached to the wrist comprises a control unit and a user interface. The control unit controls and monitors the operation of the measuring device. The necessary heart rate data processing is also carried out in the control unit. The control unit is typically a microprocessor also comprising an ROM memory in which the software of the measuring device is stored. The control unit can also comprise separate memory in which measurement data generated during the use of the device can be stored for further processing. For further processing, the data can be transferred to a separate personal computer.

The user interface of a heart rate monitor comprises selection means for making selections, and display means for displaying data. The selection means are typically push buttons. The number of buttons may vary, typically totaling three separate buttons. In addition, a so-called wireless button can be used. This means that the user selects the desired function, e.g. the start of measurement, by a special operation, e.g. by bringing the transmitter and the receiver close to one another. This closeness is detected in the magnetic coils on account of the changes which their closeness causes in the magnetic field. A conventional liquid crystal display typically serves as the display means.

The user operates the heart rate monitor by pressing the buttons. The heart rate monitor provides feedback on its display as text, numbers and various symbols.

The basic structure of the user interface in nearly all known heart rate monitors comprises different operating modes. A heart rate monitor usually comprises at least a watch mode and a heart rate measurement mode. In watch mode the heart rate monitor operates as a normal wrist watch. An operating mode may also have sub-operating modes somehow associated with the operating mode. In sub-operating modes, different parameters associated with exercising are displayed to the user. The time of day is a parameter indicating real exercise time. The date can be displayed. An alarm clock type of sub-operating mode is also common.

Different parameters measured for the exercise are displayed in heart rate measurement mode. Examples of sub-operating modes are e.g. exercise time and heart rate, real exercise time and heart rate, effective exercise time and heart rate, energy consumed by the user in the exercise and heart rate. In heart rate measurement mode the user can also be controlled by means of sound signals and symbols displayed on the display. The control may aim at keeping the exercise within effective and safe limits (typically within the range 55 to 85% of a person's maximum heart rate). In this case the user himself typically sets the lower and upper limits for his heart rate. The limits are established on the basis of information obtained in medical studies. During exercise, the measuring device gives an alarm if the heart rate exceeds the upper limit or falls below the lower limit.

The operating modes often also comprise a set mode. The set mode allows the user to set functions controlling and facilitating the exercise, e.g. said lower and upper limits for the heart rate.

The operating modes may also comprise a file mode. This is subject to the device comprising memory for storing data during exercise in the manner described above. In file mode the stored data can be studied and analyzed later.

### SUMMARY

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims, or can be learned by the practice of the principles set forth herein.

The approaches set forth herein can be used to construct a USB wrist device for sensing and calculating measurements from a user. The wrist device can be configured to be worn by the user around a wrist of the user. The measurements can include, for example, heart rate and exercise levels. The wrist device can include a glue pond containing and at least partially securing a portion of the USB connector or printed control board (PCB) on the wrist device. The glue pond can be a reservoir of glue configured to seal moisture in and from the wrist device to prevent water or fluid leakage through the USB connection. The mechanical construction of the wrist device can be used to hold the USB PCB firmly in place without moving. Moreover, the mechanical construction of the wrist device can provide a band attachment construction on a side of the wrist device housing the necessary components to establish a USB connection with a separate device. The wrist device construction can reliably secure the USB connection components on the wrist device, and prevent movement of the components or moisture damage.

Disclosed are measuring devices configured to be worn on a wrist of a user. A measuring device can include a processor, a memory, and a case including a display and the processor. The measuring device can also include aUSB plug electrically and mechanically coupled with and extending from the case, the USB plug structured and arranged to be insertable directly into a USB port of a device, the USB plug including a first coupling member. The measuring device can include first and second straps configured to secure the measuring device to the wrist of the user, the first strap including a second coupling member configured to be coupled with the first coupling member. Moreover, the measuring device can be configured to measure a physical parameter of the user via a sensor on the measuring device.

The sensor can include a heart rate monitor, a pedometer, an oximeter, an accelerometer, a gyroscope, a motion sensor, a light sensor, a camera, a temperature sensor, and so forth. Moreover, the physical parameter can include a hear rate, exercise levels, a blood pressure, a pulse, etc. In some cases, the measuring device can be a watch, for example.

### BRIEF DESCRIPTION OF THE DRAWINGS

In order to describe the manner in which the above-recited and other advantages and features of the disclosure can be obtained, a more particular description of the principles briefly described above will be rendered by reference to specific embodiments thereof which are illustrated in the appended drawings. Understanding that these drawings depict only exemplary embodiments of the disclosure and are not therefore to be considered to be limiting of its scope, the principles herein are described and explained with additional specificity and detail through the use of the accompanying drawings in which:

FIG. 1 illustrates an exemplary measuring device in accordance with various aspects of the disclosure;

FIG. 2 is an exploded view of the exemplary measuring device of FIG. 1;

FIG. 3 illustrates an exemplary open view of the measuring device of FIG. 1;

FIG. 4 illustrates a first exemplary cross-sectional view of the measuring device in FIG. 1;

FIG. 5 illustrates a second exemplary cross-sectional view of the measuring device in FIG. 1;

FIG. 6 illustrates a third exemplary cross-sectional view of the measuring device of FIG. 1;

FIG. 7 illustrates an exemplary embodiment of a measuring device; and

FIGS. 8A and FIG. 8B illustrate exemplary system embodiments.

### DETAILED DESCRIPTION OF EMBODIMENTS

In the following detailed description numerous specific details are set forth in order to provide a thorough understanding of the present invention. However, it will be understood by one skilled in the art that the present invention may be practiced without these specific details. In other instances, well known methods, procedures, and components have not been described in detail so as not to obscure aspects of the present invention.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present). Also, claim language reciting “at least one of” a set indicates that one member of the set or multiple members of the set satisfy the claim. For example, a condition requiring at least one of A and B is satisfied by any one of the following: A is true (or present) and B is false (or not present), A is false (or not present) and B is true (or present), and both A and B are true (or present).

The invention is described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. In the drawings, the size and relative sizes of layers and regions may be exaggerated for clarity.

It will be understood that when an element or layer is referred to as being “on”, “connected to” or “coupled to” another element or layer, it can be directly on, connected or coupled to the other element or layer or intervening elements or layers may be present. In contrast, when an element is referred to as being “directly on,” “directly connected to” or “directly coupled to” another element or layer, there are no intervening elements or layers present. It will further be understood that when a particular step of a method is referred to as subsequent to another step, it can directly follow said other step or one or more intermediate steps may be carried out before carrying out the particular step. Like numbers refer to like elements throughout. As used herein, the term “and/or” includes any and all combinations of one or more of the associated listed items.

It will be understood that, although the terms first, second, third, etc., may be used herein to describe various elements, components, regions, layers and/or sections. These elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

Spatially relative terms, such as “beneath”, “below”, “lower”, “above”, “upper” and the like, may be used herein for ease of description to describe one element or feature's relationship to another element(s) or feature(s) as illustrated in the figures.

It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

Embodiments of the invention are described herein with reference to cross-section illustrations that are schematic illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein. All publications, patent applications, patents, and other references mentioned herein are incorporated by reference in their entirety. In case of conflict, the present specification, including definitions, will control. In addition, the materials, methods, and examples are illustrative only and not intended to be limiting.

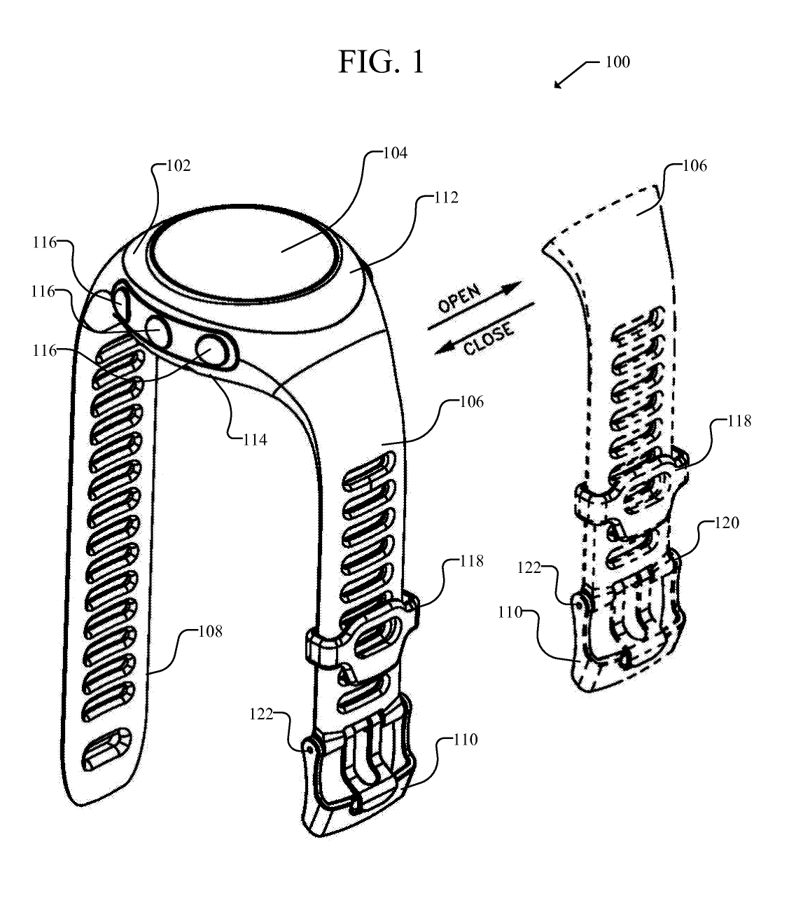

The disclosed technology addresses the need in the art for accurate and efficient measuring wrist devices. Disclosed are measuring devices configured to be worn on a user's wrist. A brief introductory description of an exemplary measuring device is disclosed herein. A detailed description of measuring devices and exemplary variations will then follow. These variations shall be described herein as the various embodiments are set forth. The disclosure now turns to FIG. 1.

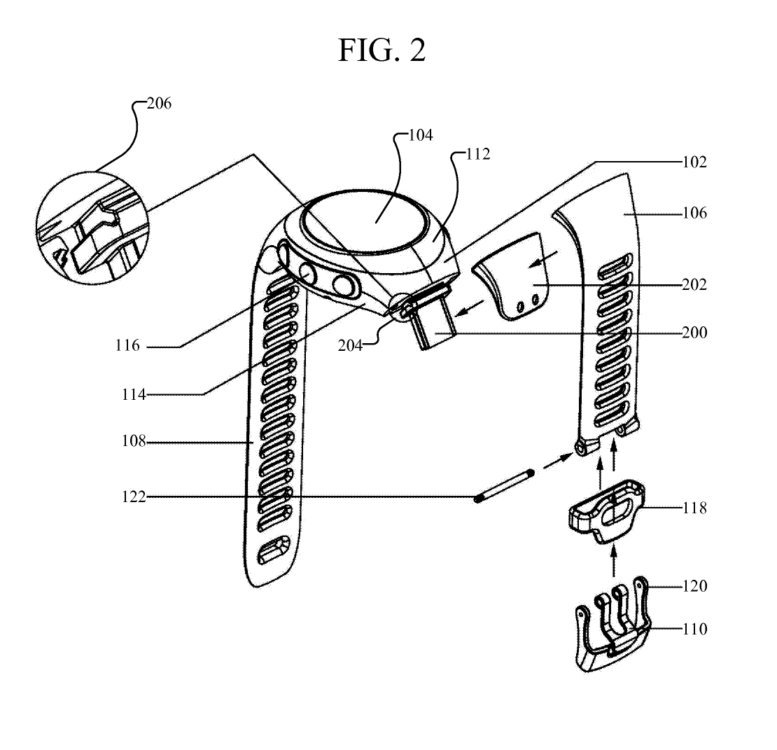

FIG. 1 schematically shows an exemplary measuring device 100 in accordance with various aspects of the disclosure. As shown, the measuring device 100 can include a case 102, a display 104, a first strap 106, and a second strap 108. Referring to FIGS. 1 and 2, the display 104 can be coupled with the case 102. The first and second straps 106, 108 can also be coupled with the case 102, at opposite sides thereof. The first and second straps 106, 108 can also be coupled with one another by a buckle 110 associated with the first strap 106. The straps 106, 108 can be, for example, belts or bands attached to the case 102 of the measuring device 100 to allow the measuring device 100 to be fit, attached, secured, and/or worn around an object, such as a user's wrist. For example, the buckle 110 on the measuring device can be configured to secure, tighten, or lock the straps 106, 108 around an object, such as a user's wrist. In some cases, the measuring device 100 can include a clasp or a fabric fastener, for example, to secure or attach the measuring device 100 around an object, instead of—or in addition to—the buckle 110. Also, the buckle 110 can include tongs 120 configured to receive a spring rod 122 for attaching or securing the buckle 110 to the first strap 106.

Moreover, the case 102 can be configured to house, secure, and/or contain one or more components of the measuring device, such as a sensor (e.g., a pedometer, a heart rate sensor, an oximeter, etc.), a battery, a spring, an antenna, an interface, a printed circuit board (PCB), a processor, a wire, a mechanical part (e.g., a rotor, a weight, a dial, a coil, etc.), a capacitor, a generator, a photovoltaic cell, a lamp, a memory, etc.

In some aspects, the display 104 can be an LCD display, an LED display, or the like. The display 104 can also be covered by a lens, such as, for example, an acrylic lens. The case 102 can include a top cover 112 and a case back 114, which can be coupled to one another by any conventional means. The measuring device 100 can include a control module (not shown), such as a PCB, disposed and mounted inside the case 102. The case 102 can also include one or more control buttons 116 associated therewith. The control buttons 116 can be configured to be electrically connected with the control module in order to control operation of the measuring device 100. The control module can include a memory configured to store user information, measurement data, sensed data, statistics, configuration settings, instructions, logic, and/or any other information, such as user information. The control module can be electrically coupled with the display 104 to generate or present a visual representation of data to a user. The control module can also be electrically coupled with any other components on the measuring device 100 or inside the case 102, such as a sensor, a battery, an antenna, an interface, etc.

Moreover, the first strap 106 can be configured to be unattached to the case 102 to open the case 102, or expose one or more components inside the case 102, such as PCB 202, as further illustrated in FIG. 2 and the accompanying discussion, below.

Referring to FIG. 2, the PCB 200 can be a communications interface or a paddle card for electrically connecting the measuring device 100 to other devices. For example, the PCB 200 can be an electrical paddle card configured for electrical mating with a corresponding, compatible connector for support electrical signaling and communications. In some embodiments, the PCB 200 can be a universal serial bus (USB) PCB for connecting the measuring device 100 to other electrical devices via the USB communications standard. Here, the measuring device 100 can be connected, via the USB PCB, to a corresponding USB socket, port, or connector on another device. The USB PCB can be configured to communicate according to any version of the USB communications standard, such as USB 1, USB 2.x, USB 3.x, or any other future version of the USB communications standard. The USB PCB can be a male USB connector configured to electrically couple to a female USB connector. Alternatively, the USB PCB can be a female USB connector configured to electrically couple to a male USB connector.

The USB PCB can be configured according to one or more class codes, and can contain specific drivers or logic for communicating with other types of devices. Moreover, the USB PCB can be configured based on any particular USB type, such as type A, type B, Mini-A, Mini-B, and so forth. To this end, the shape and dimensions of the corresponding port of the USB PCB can vary based on the USB type. In some cases, the USB PCB can include a circuit, a socket, and one or more pins, such as a power pin, a ground pin, and data pins. As one of ordinary skill in the art will readily recognize, the various pins can be configured to carry specific electrical signals.

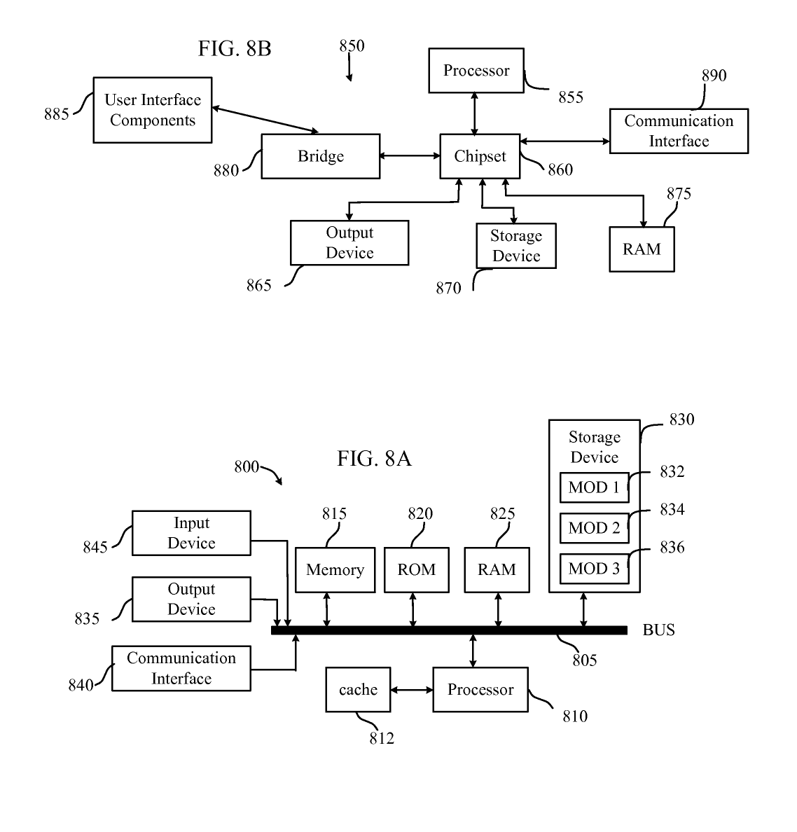

Further, the USB PCB can be configured to transmit and receive data to and from the measuring device 100. For example, the USB PCB can transmit sensed data, such as heart rate levels or exercise levels, to another device, such as a computing device as illustrated in FIG. 8. The USB PCB can also transmit and receive data and instructions, and connect the measuring device 100 to other devices, such as peripheral devices, mobile devices, or any other computing devices. In addition, the USB PCB can carry data for storage at a memory on the measuring device 100 and/or a memory on another device. Also, the USB PCB can carry power, which allows a user to transfer power from a computing device to a battery or power storage device on the measuring device 100. This way, a user can connect the measuring device 100 to another device via the USB PCB to obtain a power charge to power the measuring device 100. As previously mentioned, the power charge used by the measuring device 100 can be stored in a battery or any other power storage device.

In some cases, the USB PCB can enable a user to conveniently transfer data to and from the control module on the measuring device 100. For example, the USB PCB can allow a user to connect the measuring device 100 to a compatible device to transfer data stored on the control module to the compatible device, and vice versa. This way, the control module on the measuring device 100 can collect and store data on the measuring device 100, such as heart rate levels, exercise levels, or other physiological parameters, and transmit such data to other devices, such as a computer.

As one of ordinary skill in the art will readily recognize, the PCB 200 can be configured according to any communications protocol, such as a high-speed communications protocol for transferring data. However, the USB standard is used herein for illustration purposes.

In some aspects, the PCB 200 can be coupled with and extend from one side of the case 102. Moreover, the measuring device 100 can include a band insert 202 to attach, fit, lock, or secure the PCB 200 to the first strap 106. The band insert 202 can also serve to protect or shield the PCB 200 from external forces, for example.

As previously mentioned, the PCB 200 can be coupled to the case 102 of the measuring device 100. In some cases, the PCB 200 can be electrically coupled to the control module on the measuring device 100. The PCB 200 can be electrically coupled at connection point 204. The exploded view 206 illustrates an example of the connection point 204 which can be used to electrically connect the PCB 200 to the control module or any other electrical component in the measuring device 100.

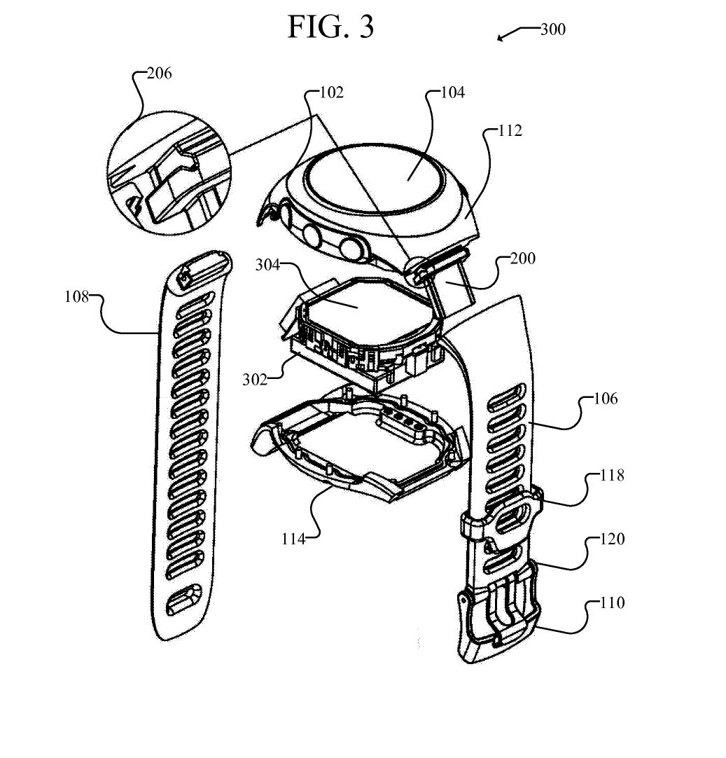

FIG. 3 illustrates an exemplary open view 300 of the measuring device 100 in FIG. 1. As illustrated, the top cover 112 and the back cover 114 of the measuring device 100 can be separated or opened to allow access to the components inside of the case 102 of the measuring device. The case 102 can house module 302 which can hold, secure, fit, or maintain any of the internal components of the measuring device 100. In some aspects, the module 302 can hold the control module 304 which, as previously explained, can be an electrical board configured support one or more functions, such as calculations, storage functions, measurements, etc. The inside case 102, can also hold other components, such as sensors, batteries, memory, etc.

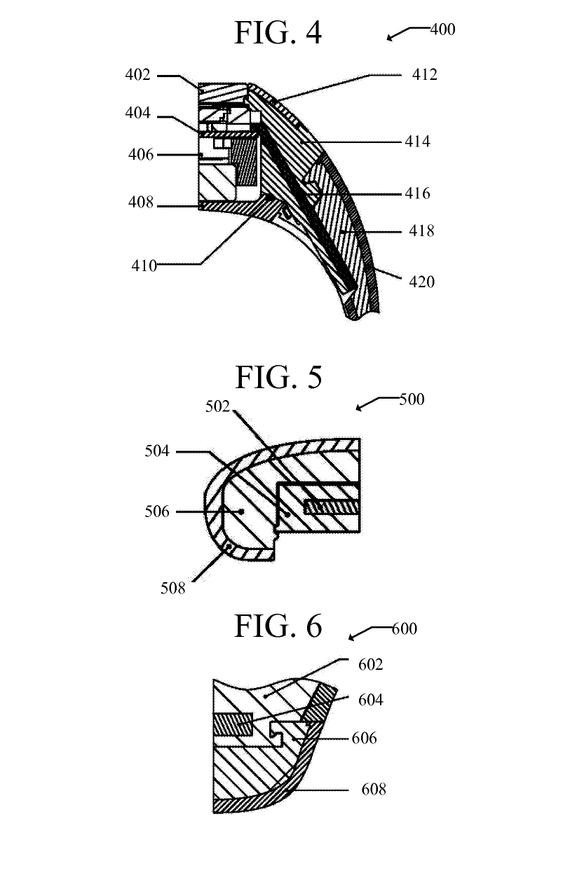

FIG. 4 illustrates a first exemplary cross-sectional view 400 of the measuring device of FIG. 1. The lens 402 is positioned or coupled to the top cover 412 and enclosed by the case back 408 of the measuring device 100. In some cases, the lens 402 can be an acrylic lens, for example. The case 414 can also enclose the control module 404 and the module 406, which can be positioned between the lens 402, the top cover 412, and/or the case back 408. The control module 404 can be, for example, a PCB for controlling the functions of the measuring device 100. The case 414 can include a coupling member configured to receive a complimentary shaped and sized coupling member of the first strap 106.

The USB PCB 416 can be coupled to the control module 404 and held or secured by the band insert 418 of the measuring device 100. The band insert 418 can also be held or secured by the band 420 of the measuring device 100.

The measuring device 100 can include a D-ring 410 to secure or hold any of the components of the device 100. In some cases, the D-ring 410 can be a silicone attachment device or clasp, for example.

FIG. 5 illustrates a second exemplary cross-sectional view 400 of the measuring device of FIG. 1. As illustrated, the USB PCB 502 can be attached to the case 504 of the measuring device 100. The band insert 506 can be sized and arranged to removably receive the USB PCB 502. Moreover, the band insert 506 can be attached or coupled to the band 508.

FIG. 6 illustrates a third exemplary cross-sectional view 400 of the measuring device of FIG. 1. The case 602 can be sized and arranged to removably receive the USB PCB 604. Here, the case 602 can secure, hold, lock, attach, and/or protect the USB PCB 502. The band insert 606 can also be sized and arranged to removably receive the USB PCB 604. Thus, the USB PCB 604 can be secured in the measuring device using the case 602 and the band insert 606. The connection point can be configured to be secure yet flexible to protect the USB PCB 604 from external forces, rotation, and otherwise provide strain relief. The band insert 606 can be removably sized, arranged, and secured to the band 608.

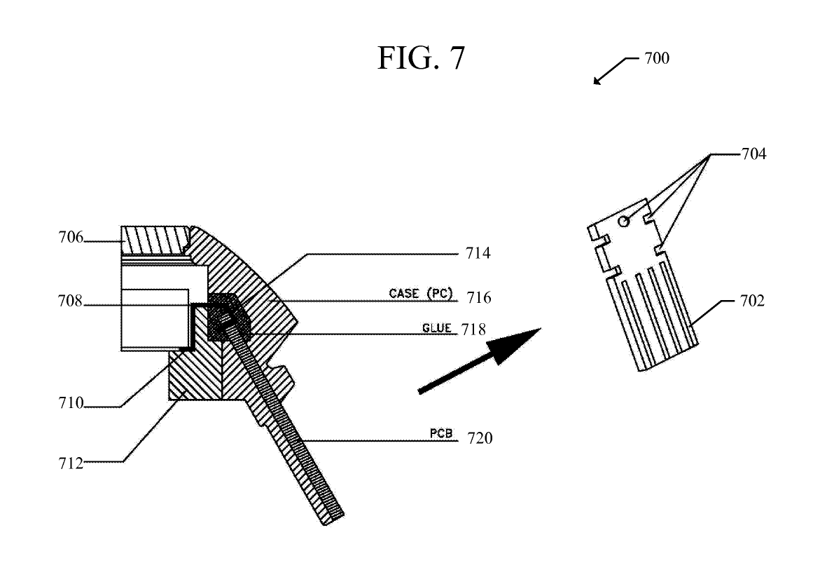

FIG. 7 illustrates an exemplary embodiment of a measuring device 700. The measuring device 700 can be similar to the measuring device 100 shown in FIG. 1. The measuring device 700 can include a lens which can be arranged to cover a display such as an LED or LCD. The case 716 and case back 712 can house the components of the computing device 700. The PCB 720 can be coupled to the control module via an enameled wire 708. The enameled wire 708 can help lock or hold the PCB 720 securely in place. The case 716 can also include a cavity 714 which can be a reservoir to house or receive glue for further securing the enameled wire 708 and the PCB 720 in place. The glue in the cavity 714 can provide a seal to protect the PCB 720, and any other inside component, from water and moisture. Moreover, the glue in the cavity 714 can help lock the PCB 720 and prevent movement to avoid damage to the circuits or connection elements in the PCB 720. In some aspects, the glue in the cavity 714 can also provide strain relief against external forces. The glue can include silicone, plastic, rubber, diecast, or any other glue substance according to conventional methods.

The measuring device 700 can include a slot 710 for receiving a portion of the enameled wire 708 and/or to further secure the enameled wire 708 to the control module, the case back 712, and/or the case 716.

The measuring device 700 can include a PCB 702 for connecting the measuring device 700 to a separate device and establishing a communication with the separate device. The PCB 702 can include slots and protrusions 704 for coupling or attaching the PCB 702 to the computing device 700. In some aspects, the protrusions 704 can include an opening for receiving or inserting the enamelled wire 708 through the protrusion on the PCB 702.

Devices of the present disclosure are suitable for use in all types of measuring devices, which are carried by a user during exercise for measuring, non-invasively, at least one signal from the body, for example, in heart rate monitors, and even in advanced versions of heart rate monitors in which, for example, the user's energy consumption, blood pressure, etc., are measured in addition to, or instead of, the heart rate.

In one exemplary embodiment of the present disclosure, the measuring devices 100 and 700 can include a heart rate monitor. Moreover, the control module of the heart rate monitor can include a measuring unit, as well as a control unit for controlling the measuring unit. The control module can also control a user interface including the control buttons 116 and display 104. The control unit can include a PCB or a microprocessor, for example. The control unit can also include a memory medium, such as ROM memory, in which software or logic controlling the device can be stored, as well as any other data, instructions, or measurements. The control module can further include additional memory in which information on, for example, heart rate gathered during the measurement can be stored. In some cases, the control unit can also be implemented by an ASIC circuit or another coupling composed of HW units. Thus, the measuring device 100, 700 can include variations of software and/or hardware components.

The measuring unit can be one piece, for example, a heart rate monitor carried on the wrist. The heart rate can be measured from the wrist using sensed data and any other data, such as user inputs, which can be provided via the controls 116 for example. In some aspects, a measurement result can be obtained by the present technology using a solution of the type described herein, in which the measuring unit is divided into two parts. For example, the measuring unit can be divided into a wireless transmitter that is attached around the chest of the user and which measures the user's heart rate, and a heart rate receiver in the measuring device 100, 700 attached to the user's wrist.

In various aspects, the display 104 can display the time of day and/or the date, when in watch mode. The display 104 can also display a heart symbol indicating whether heart rate measurement is active or inactive. The control buttons 116 shown in FIG. 1 can provide a user input or selection means for shifting between different modes, displays, or content. For example, one button may be used for making selections and for starting and stopping functions. One button may be intended for adjusting the settings and for using a background light of the display 104 and a sound signal during a measurement function. The control buttons 116 can also be configured to allow the user to select content, modify settings, initiate functions, modify functions, enter parameters, stop functions, manage the measuring device 100, 700, browse through information on the display 104, control the presentation of information, and otherwise program or control the measuring device 100, 700.

The measuring device 100, 700 can allow the user, for example, to program a shortcut function for a selection means in set mode. This short-cut allows the user to rapidly access a frequently used function. Another feature facilitating the use is a home selection function that allows the user to rapidly access the basic mode of the device, e.g. watch mode. Both the short-cut and home selection can be implemented in various ways, as would be appreciated by persons skilled in the art. An alternative is to simultaneously press several buttons, for example, two different buttons, to make the selection. Another alternative is to press the button for an extended period of time, for example, two seconds, after which the selection is made.

FIGS. 8A and 8B illustrate exemplary possible system embodiments. The system embodiments can illustrate the various components in the measuring devices 100 and 700 or a separate device which can be electrically connected to the measuring devices 100 and 700 via the USB PCB on the measuring devices 100 and 700. The more appropriate embodiment will be apparent to those of ordinary skill in the art when practicing the present technology. Persons of ordinary skill in the art will also readily appreciate that other system embodiments are possible.

FIG. 8A illustrates a conventional system bus computing system architecture 800 wherein the components of the system are in electrical communication with each other using a bus 805. Exemplary system 800 includes a processing unit (CPU or processor) 810 and a system bus 805 that couples various system components including the system memory 815, such as read only memory (ROM) 820 and random access memory (RAM) 825, to the processor 810. The system 800 can include a cache of high-speed memory connected directly with, in close proximity to, or integrated as part of the processor 810. The system 800 can copy data from the memory 815 and/or the storage device 830 to the cache 812 for quick access by the processor 810. In this way, the cache can provide a performance boost that avoids processor 810 delays while waiting for data. These and other modules can control or be configured to control the processor 810 to perform various actions. Other system memory 815 may be available for use as well. The memory 815 can include multiple different types of memory with different performance characteristics. The processor 810 can include any general purpose processor and a hardware module or software module, such as module 1 832, module 2 834, and module 3 836 stored in storage device 830, configured to control the processor 810 as well as a special-purpose processor where software instructions are incorporated into the actual processor design. The processor 810 may essentially be a completely self-contained computing system, containing multiple cores or processors, a bus, memory controller, cache, etc. A multi-core processor may be symmetric or asymmetric.

To enable user interaction with the computing device 800, an input device 845 can represent any number of input mechanisms, such as a microphone for speech, a touch-sensitive screen for gesture or graphical input, keyboard, mouse, motion input, speech and so forth. An output device 835 can also be one or more of a number of output mechanisms known to those of skill in the art. In some instances, multimodal systems can enable a user to provide multiple types of input to communicate with the computing device 800. The communications interface 840 can generally govern and manage the user input and system output. There is no restriction on operating on any particular hardware arrangement and therefore the basic features here may easily be substituted for improved hardware or firmware arrangements as they are developed.

Storage device 830 is a non-volatile memory and can be a hard disk or other types of computer readable media which can store data that are accessible by a computer, such as magnetic cassettes, flash memory cards, solid state memory devices, digital versatile disks, cartridges, random access memories (RAMs) 825, read only memory (ROM) 820, and hybrids thereof.

The storage device 830 can include software modules 832, 834, 836 for controlling the processor 810. Other hardware or software modules are contemplated. The storage device 830 can be connected to the system bus 805. In one aspect, a hardware module that performs a particular function can include the software component stored in a computer-readable medium in connection with the necessary hardware components, such as the processor 810, bus 805, display 835, and so forth, to carry out the function.

FIG. 8B illustrates a computer system 850 having a chipset architecture that can be used in executing the described method and generating and displaying a graphical user interface (GUI). Computer system 850 is an example of computer hardware, software, and firmware that can be used to implement the disclosed technology. System 850 can include a processor 855, representative of any number of physically and/or logically distinct resources capable of executing software, firmware, and hardware configured to perform identified computations. Processor 855 can communicate with a chipset 860 that can control input to and output from processor 855. In this example, chipset 860 outputs information to output 865, such as a display, and can read and write information to storage device 870, which can include magnetic media, and solid state media, for example. Chipset 860 can also read data from and write data to RAM 875. A bridge 880 for interfacing with a variety of user interface components 885 can be provided for interfacing with chipset 860. Such user interface components 885 can include a keyboard, a microphone, touch detection and processing circuitry, a pointing device, such as a mouse, and so on. In general, inputs to system 850 can come from any of a variety of sources, machine generated and/or human generated.

Chipset 860 can also interface with one or more communication interfaces 890 that can have different physical interfaces. Such communication interfaces can include interfaces for wired and wireless local area networks, for broadband wireless networks, as well as personal area networks. Some applications of the methods for generating, displaying, and using the GUI disclosed herein can include receiving ordered datasets over the physical interface or be generated by the machine itself by processor 855 analyzing data stored in storage 870 or 875. Further, the machine can receive inputs from a user via user interface components 885 and execute appropriate functions, such as browsing functions by interpreting these inputs using processor 855.

It can be appreciated that exemplary systems 800 and 850 can have more than one processor 810 or be part of a group or cluster of computing devices networked together to provide greater processing capability.

For clarity of explanation, in some instances the present technology may be presented as including individual functional blocks including functional blocks comprising devices, device components, steps or routines in a method embodied in software, or combinations of hardware and software.

In some embodiments the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer readable media. Such instructions can comprise, for example, instructions and data which cause or otherwise configure a general purpose computer, special purpose computer, or special purpose processing device to perform a certain function or group of functions. Portions of computer resources used can be accessible over a network. The computer executable instructions may be, for example, binaries, intermediate format instructions such as assembly language, firmware, or source code. Examples of computer-readable media that may be used to store instructions, information used, and/or information created during methods according to described examples include magnetic or optical disks, flash memory, USB devices provided with non-volatile memory, networked storage devices, and so on.

Devices implementing methods according to these disclosures can comprise hardware, firmware and/or software, and can take any of a variety of form factors. Typical examples of such form factors include laptops, smart phones, small form factor personal computers, personal digital assistants, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

Although a variety of examples and other information was used to explain aspects within the scope of the appended claims, no limitation of the claims should be implied based on particular features or arrangements in such examples, as one of ordinary skill would be able to use these examples to derive a wide variety of implementations. Further and although some subject matter may have been described in language specific to examples of structural features and/or method steps, it is to be understood that the subject matter defined in the appended claims is not necessarily limited to these described features or acts. For example, such functionality can be distributed differently or performed in components other than those identified herein. Rather, the described features and steps are disclosed as examples of components of systems and methods within the scope of the appended claims.

In the claims the word “comprising” does not exclude other elements or steps, and the indefinite article “a” or “an” does not exclude a plurality. A single component or other unit may fulfil the functions of several items recited in the claims. The mere fact that certain measures are recited in mutually different claims does not indicate that a combination of these measures cannot be used to advantage. Any reference signs in the claims should not be construed as limiting the scope.

What is claimed is:

1. A measuring device configured to be worn on a wrist of a user, the measuring device comprising:
a processor;
a computer-readable storage medium for storing at least one of data, measurements, and instructions;
a case comprising a display and the processor;
a USB plug electrically and mechanically coupled with and extending from the case, the USB plug structured and arranged to be insertable directly into a USB port of a device, the USB plug comprising a first coupling member; and
first and second straps configured to secure the measuring device to the wrist of the user, the first strap comprising a second coupling member configured to be coupled with the first coupling member.

2. The measuring device of claim 1, further comprising a band insert configured to be coupled to the USB plug and a corresponding strap comprising one of the first and second straps.

3. The measuring device of claim 1, wherein the USB plug comprises a USB PCB and wherein the measuring device comprises a watch.

4. The measuring device of claim 3, further comprising a mechanical lock configured to hold the USB PCB and reduce movement of the USB PCB.

5. The measuring device of claim 3, further comprising a cavity provided inside of the case and configured to receive at least a portion of the USB PCB.

6. The measuring device of claim 5, wherein the cavity is further configured to receive glue to at least partially secure the USB PCB in an attached position.

7. The measuring device of claim 6, the cavity comprising a glue pong configured to seal a USB connection to prevent water leakage.

8. The measuring device of claim 1, wherein the USB plug comprises an electrical paddle card configured to connect to a compatible connector on a separate device.

9. The measuring device of claim 1, wherein the USB plug is configure to establish electrical mating with the USB port on the device, the device comprising one of a mobile device, a computer, or a server.

10. The measuring device of claim 1, further comprising at least one PCB for performing operations based on instructions stored on the computer-readable storage medium.

11. The measuring device of claim 1, further comprising a sensor, the sensor comprising at least one of a heart rate monitor, a pedometer, an oximeter, an accelerometer, a gyroscope, a motion sensor, a light sensor, a camera, and a temperature sensor.

12. The measuring device of claim 1, further comprising a control module electrically coupled to the USB plug.

13. The measuring device of claim 1, further comprising an enamelled wire coupled to at least a portion of the USB plug.

14. The measuring device of claim 1, wherein the enamelled wire is configured to be inserted through an opening on the USB plug, the measuring device further comprising a lens.

15. An apparatus comprising:
a wrist device configured to be worn on a wrist of a user, the wrist device comprising:
a PCB;
a display;
a sensor;
a case housing the at least one of the PCB, the memory, the display, the sensor, and an electrical paddle card configured to be insertable into a mating connector to form a mating connection, the case comprising a cavity housing at least a portion of the electrical paddle card, wherein the cavity is filled with a glue material; and
first and second bands configured to attach the wrist device to the wrist of the user.

16. The apparatus of claim 15, further comprising a memory configured to store data associated with the wrist device.

17. The apparatus of claim 15, wherein the sensor comprises at least one of a heart rate monitor, a pedometer, an oximeter, an accelerometer, a gyroscope, a motion sensor, a light sensor, a camera, and a temperature sensor, and wherein the electrical paddle card comprises a USB PCB.

18. The apparatus of claim 17, further comprising a control button configured to receive input from the user for controlling an operation of the wrist device via the PCB.

19. A method comprising:
receiving sensed data associated with a user, the sensed data being received via a sensor on a wrist device, the wrist device comprising a control module and a USB PCB, wherein the wrist device is configured to attach around a wrist of the user, and wherein at least a portion of the USB PCB is contained within a cavity of the wrist device, the cavity being filled with a glue material; and
calculating a physical parameter of the user via the sensor on the wrist device; and
sending the physical parameter to a separate device via the USB PCB.

20. The method of claim 19, wherein the sensor comprises a heart rate monitor and the physical parameter comprises a heart rate, and wherein the physical parameter is sent to the separate device via a USB connection established with the separate device via the USB PCB, the method further comprising presenting the physical parameter via a display on the wrist device.
